(19) United States
(12) Patent Application Publication
(10) Pub. No.: US 2009/0286237 A1
(43) Pub. Date: Nov. 19, 2009

(54) Diagnostic Kits and Methods for Oesophageal Abnormalities

(75) Inventors: Rebecca C. Fitzgerald, Cambridge (GB); Richard Hardwick, Cambridge (GB)

Correspondence Address: EDWARDS ANGELL PALMER & DODGE LLP, P.O. BOX 55874, BOSTON, MA, 02205, US

(73) Assignees: Medical Research Council, London (GB); Cambridge Enterpriese, Cambridge (GB)

(21) Appl. No.: 12/083,751

(22) PCT Filed: Oct. 20, 2006
(86) PCT No.: PCT/GB2006/003913

§ 371 Date: May 19, 2009

(30) Foreign Application Priority Data
Oct. 21, 2005 (GB) 0521521.5

(51) Int. Cl. C12Q 1/68; C12Q 1/02

(52) U.S. Cl. 435/6; 435/29

(57) ABSTRACT

The invention relates to kits and methods for aiding the diagnosis of Barrett's oesophagus or Barrett's associated dysplasia. Preferred is a method comprising assaying cells from the surface of a subject's oesophagus for a non-squamous cellular marker, wherein detection of such a marker indicates increased likelihood of the presence of Barrett's or Barrett's associated dysplasia, preferably wherein said sample of cells is not directed to a particular site within the oesophagus. The invention also encompasses a method comprising sampling the cellular surface of the oesophagus of said subject. The invention also relates to a kit comprising a swallowable device comprising abrasive material capable of collecting cells from the surface of the oesophagus, together with printed instructions for its use in detection of Barrett's oesophagus or Barrett's associated dysplasia. Preferably said device comprises a capsule sponge.

### FIELD OF THE INVENTION

The invention relates to methods for detection of oesophageal abnormalities such as Barrett's oesophagus and Barrett's associated dysplasia including adenocarcinoma. Furthermore, the invention relates to kits for sampling oesophageal cells and detecting cellular markers associated with the above conditions.

### BACKGROUND TO THE INVENTION

Oesophageal adenocarcinoma is rapidly increasing and is preceded by a condition called Barrett's oesophagus. Early diagnosis is crucial to improving the appalling outcome (>80% mortality at 5 years) from oesophageal adenocarcinoma. Currently the majority of patients with Barrett's oesophagus remain undiagnosed in the population. Furthermore, of those that are diagnosed it is not currently possible to accurately predict the small proportion (1% per year) that will progress to cancer. It has been suggested that endoscopic screening should be performed to detect those at risk for Barrett's oesophagus—for example endoscopy for males over 50 yrs with chronic heartburn symptoms. This is too costly and demanding on service resources.

Endoscopy is a very invasive technique. Patients need to be sedated and have local anaesthetic. Endoscopy requires a trained endoscopist accompanied by two nurses. Furthermore, endoscopic biopsies need to be processed in a laboratory for analysis, which requires all experienced histologist to examine the sections. Thus it can be appreciated that endoscopy causes patient discomfort, and is extremely expensive in terms of the equipment and the resources and staffing levels needed to carry out the procedures.

Endoscopy carries a 1 in 10,000 risk of death and a 1 in 1,000 risk of complication such as bleeding or perforation. Although this may appear statistically low, the complications are particularly dangerous. One complication can be oesophageal bleeding from the sites of the biopsies. This bleeding can be so severe as to require transfusion and so represents a serious risk to the individual being examined. The second risk from endoscopic biopsy is a risk of perforation of the oesophagus. If this occurs, the patient is likely to proceed immediately into theatre for oesophageal surgery. This is very serious, not only due to requiring a general anaesthetic but also due to the severity of surgical procedures on the oesophagus. Even if endoscopy proceeds without either of these dangerous complications, the patient requires time to recover. Even to have the procedure will require a day off work for the patient.

The gold standard in diagnosing oesophageal adenocarcinoma or indeed dysplasia which can lead to the same is by extraction of a deep cell sample preserving the cellular architecture. As noted above, this is performed by endoscopy. In order to assess dysplasia, the histologist must look at the whole section and score it on a number of factors. These factors include nuclear crowding and a range of morphological characteristics across the whole depth of the tissue. One criterion is that the dysplasia must extend to the surface of the tissue sample, and so a full depth section is needed in order to assess whether this has occurred. Once tissue architecture has been lost, cytologists generally cannot tell which cells are columnar, which represent Barrett's or which are dysplastic. Furthermore, if inflammatory cells are present such as lymphocytes, when tissue architecture has been lost there is no positional information to give a clue as to whether such cells have migrated to a site important for assessment of dysplasia, or whether they have been collected from some other minor injury for example created by swallowing a bone or other sharp object in a foodstuff. Thus, an intact tissue architecture is generally regarded as essential for the accurate assessment of a patient's condition.

An alternative technique has been to collect endoscopic brushings. This involves all of the risks outlined above concerning endoscopic collection of biopsies. Although endoscopic brushing has the advantage that the sample collection can be directed to the site of Barrett's oesophagus, the amount of cellular material obtained is very small. The endoscopic brush head is constrained in size since it must fit down the endoscope channel. Furthermore, it requires a skilled operator even to perform endoscopic brushing. The tolerances for correctly performing the brushing are extremely narrow. If the brushing is too vigorous, then reticulocyte contamination can obscure meaningful analysis of the sample. However, if the brushing is too light then insufficient cellular material can be collected for a meaningful analysis. Thus, the skills required to complete a satisfactory endoscopic brushing are even rarer than those required of an endoscopist.

When employing endoscopic biopsies, approximately 4-20 biopsies are collected for analysis in any one procedure. Despite this number of biopsies, still only approximately 1% of the surface area of the oesophagus is sampled in this procedure. Moreover, if the site of the dysplasia within the Barrett's oesophagus is spatially missed during the sampling procedure, then a false negative result could easily be achieved. This is clearly a significant risk factor for the patient being assessed.

Emerging technologies such as camera capsules have been considered for assessment or surveillance of Barrett's oesophagus. Camera capsules are small pill sized objects which are capable of collecting images as they pass through the alimentary canal. However, the capsules pass very quickly through the oesophagus and so the opportunity to collect images during passage through the oesophagus is very limited. Furthermore, the camera capsules are unidirectional. Therefore, as they tumble and turn on their way down the oesophagus, they can only sample a very narrow strip of tissue on the inside of the oesophagus. Furthermore, this sampling is effectively random as it is determined by the tumbling motion of the camera on its journey through the oesophagus. Thus, if the camera happens to be pointing away from the Barrett's oesophagus as it travels though the patient, then it will not be possible to collect any meaningful information for that patient. This again can lead to false negative diagnosis. Furthermore, no sample collection is possible with this approach.

Another further development is the use of nasal endoscopy. This is a miniaturised form of endoscope which can be conducted through the patient's nasal passages, rather than requiring the more invasive buccal entry endoscope. However, nasal endoscopy is of such reduced size that sample collection is not possible. Thus, no biopsies or brushings can be collected, and the technique is limited to observation only. This is clearly unsatisfactory in producing a robust diagnosis.

Another development has been the use of ‘cytomesh’ produced by Boston Scientific Inc, the Brandt balloon, and the ‘Cell-Mate’ sponge of U.S. Pat. No. 4,735,214. However, each of these approaches employs the use of rod-like delivery devices. These resemble rigid endoscopy and are potentially even more awkward than current flexible endoscopy techniques. The devices are difficult or impossible to swallow. The devices often have to be forcibly introduced into the subject and thus cause considerable discomfort or distress, as well as technical problems such as fouling on the windpipe. Thus, these are expensive and require technical expertise comparable to endoscopy in order to carry them out.

The present invention seeks to overcome problems associated with the prior art.

### SUMMARY OF THE INVENTION

The invention is based on the surprising finding that surface sampling of the oesophagus combined with cytological analysis can lead to a very robust method for diagnosing and grading oesophageal lesions such as Barrett's oesophagus, dysplasia or indeed adenocarcinoma.

The view in the art is that tissue architecture needs to be preserved in order to have a meaningful diagnosis. However, the present inventors have surprisingly shown that sampling cells from the surface of the oesophagus and analysing them cytologically, for example for markers of proliferation, can provide a very specific and sensitive technique for diagnosing and/or grading oesophageal abnormalities.

In particular, the invention provides the use of a swallowable abrasive sampling device which is introduced into the patient without the need for sedation or anaesthetic, and is withdrawn bringing with it a sample of the surface cells of the oesophagus. From an understanding of the prior art, this would have been thought unworkable for several reasons. Firstly, a small Barrett's oesophagus lesion occupies only about 1-2 cm of a 40 cm oesophagus. Thus, the expectation would be that 95-98% of the cells or even more would be squamous cells from the oesophagus, and only a few percent of the total cell sample retrieved could be expected to represent a sampling of the Barrett's oesophagus. However, the present inventors have surprisingly found that surface dysplastic cells of Barrett's oesophagus can slough off the lesion more easily than the squamous cells can slough off the intact oesophagus surface. Therefore, the methods of the present invention provide an inherent bias towards the productive sampling of the oesophageal lesion. Secondly, the prior art teaches the importance of the tissue architecture in a meaningful readout of diagnosis. Therefore, it is not expected that a mere sampling of the surface cells of the oesophagus can provide a diagnostically useful cell sample. However, the present inventors have surprisingly found that in fact a surface sample of oesophageal tissue, if analysed according to the methods of the present invention, can provide the necessary information for a robust, sensitive, specific and reliable diagnosis. Thirdly, prior art techniques involve morphological analysis of biopsies or collected cell material. Changes in cell shape and comparisons with unaffected cell morphology are required. By contrast, it is unexpectedly shown by the present inventors that an absolute readout ie. finding a particular molecular marker in a surface sample of the oesophagus can be indicative of particular disorders, without the need to relate the findings to the neighbouring cells, which would of course require intact tissue structure and cannot be performed by a population cell sampling technique. Fourth, when assessing dysplasia in the art, a whole section has to be assessed. There are many histological criteria which are applied such as nuclear crowding, depth of tissue etc., and extension of the lesion to the internal surface of the oesophagus is only one among many criteria which must be met before pronouncing a diagnosis of dysplasia. Many attempts have been made in the art to base a diagnostic method for Barrett's abnormalities on cytology, but they have failed. It is therefore unexpected that the present inventors have been able to design a scheme based on surface cell sampling in combination with molecular marker detection which provides a reliable tool for aiding diagnosis of Barrett's and related abnormalities. Fifth, the sampling techniques of the present invention are performed ‘blind’ in that no visual inspection takes place. In other words, sample collection is not directed to a particular part of the oesophagus. This is a departure from the prior art techniques which are all directed by the operator to the visible area(s) of Barrett's on the inner surface of the oesophagus.

Thus, the present invention is based upon a novel surface sampling approach to the diagnosis of Barrett's oesophagus. In particular, the invention is based upon the molecular cytological analysis of marker expression in surface sampled cells. By contrast, the prior art has been mainly concerned with histological analysis involving observation of cells at different layers within a tissue section. By advantageously combining cellular marker analysis with the surface sampling technique, the need for risky, invasive and often distressing prior art techniques such as endoscopic biopsy can be advantageously avoided.

Thus in a broad aspect the invention relates to the application of molecular biomarkers to material collected from a non-endoscopic sampling device in the diagnosis of Barrett's and Barrett's associated dysplasia including adenocarcinoma.

Thus, in one aspect the invention provides a method for aiding the diagnosis of Barrett's oesophagus or Barrett's associated dysplasia in a subject, said method comprising sampling the cellular surface of the oesophagus of said subject, and assaying the cells for a non-squamous cellular marker, wherein detection of such a marker indicates increased likelihood of the presence of Barrett's or Barrett's associated dysplasia. Preferably said sampling is not directed to a particular site within the oesophagus.

Preferably only the surface of the oesophagus is sampled. This has the advantage of avoiding more invasive sampling techniques such as biopsy collection techniques which penetrate below the surface of the oesophagus.

In another aspect, the invention provides a method for aiding the diagnosis of Barrett's oesophagus or Barrett's associated dysplasia comprising assaying cells from the surface of a subject's oesophagus for a non-squamous cellular marker, wherein detection of such a marker indicates increased likelihood of the presence of Barrett's or Barrett's associated dysplasia. In this embodiment, preferably the actual sampling of the cells is not part of the method of the invention.

Preferably the method of the invention is conducted in vitro.

Preferably the non-squamous cellular marker is a marker of cellular proliferation.

Preferably the non-squamous cellular marker is a marker of columnar cells.

Preferably the marker is selected from the group consisting of brush border proteins such as villin or moesin, mucin genes, brush border enzymes such as alkaline phosphatase, homeobox genes such as Cdx1 and/or Cdx2, cytokeratins such as CK8/18 for columnar cells, or any marker known to be differentially expressed in Barrett's versus normal oesophageal surface cells.

Preferably the marker is selected from the group consisting of proliferation markers such as Ki67 and Mcm proteins, proliferation and DNA damage markers such as PCNA, cyclins such as cyclin D and/or cyclin A, abnormal p53, loss of p16, aneuploidy or any marker known to correlate with the degree of dysplasia. More preferably the marker is Mcm2 or Cyclin A. Preferably the marker is Cyclin A. Even more preferably both Mcm2 and Cyclin A are assayed.

In another aspect, the invention provides a method as described above wherein sampling the cellular surface of the oesophagus comprises the steps of

(i) introducing a swallowable device comprising abrasive material capable of collecting cells from the surface of the oesophagus into the subject,
(ii) retrieving said device by withdrawal through the oesophagus, and
(iii) collecting the cells from the device.

Preferably step (i) comprises introducing a swallowable device comprising abrasive material capable of collecting cells from the surface of the oesophagus into the subject's stomach.

In another aspect, the invention provides a method as described above further comprising analysing the chromosomal composition of the cells, wherein detection of abnormal karyotype indicates an increased likelihood of dysplasia.

In another aspect, the invention provides a method as described above further comprising analysing the p53 status of the cells, wherein detection of abnormal p53 status indicates an increased likelihood of dysplasia.

In another aspect, the invention provides a kit comprising a swallowable device comprising abrasive material capable of collecting cells from the surface of the oesophagus, together with printed instructions for its use in detection of Barrett's oesophagus or Barrett's associated dysplasia.

In another aspect, the invention provides a kit as described above further comprising a local anaesthetic. Preferably said local anaesthetic is a spray or lozenge, preferably a spray.

In another aspect, the invention provides a kit as described above further comprising a container for receiving said swallowable device after withdrawal, said container having a quantity of preservative fluid therein. Preferably the container is a watertight container. Preferably the preservative fluid is a cell preparation fluid. Preferably said fluid is thin preparation fluid for production of slides for examination of the sampled cells.

In another aspect, the invention provides a kit as described above wherein said device comprises a capsule sponge.

In another aspect, the invention provides a kit as described above wherein said swallowable device comprises withdrawal means such as string.

In another aspect, the invention provides a kit as described above further comprising a device for severing said withdrawal means. Preferably said device comprises a blade or scissors.

In another aspect, the invention provides a kit as described above further comprising a container for administering drinkable fluid, such as water, to the subject.

In another aspect, the invention provides a kit as described above further comprising gloves. These advantageously protect the sample from contamination upon withdrawal of the device.

In another aspect, the invention provides a kit comprising a swallowable device comprising abrasive material capable of collecting cells from the surface of the oesophagus, together with printed instructions for its use in detection of Barrett's oesophagus or Barrett's associated dysplasia. Preferably said device comprises a capsule sponge.

Preferably said kit further comprises reagent for use in the detection of a non-squamous cellular marker. Preferably said non-squamous cellular marker is a marker of cellular proliferation. Preferably the non-squamous cellular marker is a marker of columnar cells.

In another aspect, the invention provides a kit as described above further comprising reagents for use in the detection of at least one marker selected from the group consisting of brush border proteins such as villin or moesin, mucin genes, brush border enzymes such as alkaline phosphatase, homeobox genes such as Cdx1 and/or Cdx2, cytokeratins such as CK8/18 for columnar cells, or any marker known to be differentially expressed in Barrett's versus normal oesophageal surface cells.

In another aspect, the invention provides a kit as described above further comprising reagents for use in the detection of at least one marker selected from the group consisting of proliferation markers such as Ki67 and Mcm proteins, proliferation and DNA damage markers such as PCNA, cyclins such as cyclin D and/or cyclin A, abnormal p53, loss of p16, aneuploidy or any marker known to correlate with the degree of dysplasia. Preferably said marker is Cyclin A.

Preferably said marker is a lectin.

In another aspect, the invention provides a kit further comprising a watertight container and preservative fluid. Preferably said fluid is for liquid based cytology, preferably said fluid is commercially available thin preparation fluid for production of slides for examination of the sampled cells.

In another aspect, the invention provides a kit as described above further comprising a local anaesthetic spray or lozenge.

In another aspect, the invention provides use of a capsule sponge in the diagnosis of Barrett's oesophagus or Barrett's associated dysplasia.

### Barrett's Oesophagus and Dysplasia

Barrett's oesophagus can occur without dysplasia. Approximately 1% of patients with Barrett's oesophagus will develop dysplasia each year. At any given time, approximately 20% of patients with Barrett's oesophagus will have dysplasia. Cancer such as adenocarcinoma develops from dysplasia and is regarded as one extreme form of dysplasia, even though pathologically the conditions clearly differ. The present invention is concerned with the detection and diagnosis of these disorders and as such adenocarcinoma is regarded as one extreme form of dysplasia, and its detection and diagnosis forms a part of the present invention as discussed herein.

Thus it can be appreciated that the invention is concerned with detection and diagnosis of a single progressive disease state that has recognisable discrete stages. These stages comprise Barrett's oesophagus, Barrett's oesophagus associated dysplasia including adenocarcinoma, which arises therefrom.

The normal state of the cells in the oesophagus is that of squamous epithelium. In Barrett's oesophagus, these cells take on the characteristics of columnar epithelium and undergo further changes as they progress through the disease states outlined above. Thus, non-squamous cells in the oesophagus are abnormal and correlate with Barrett's oesophagus and potentially with dysplasia and more serious abnormalities as discussed herein.

Consistent with the failures in the prior art, the present inventors have shown that cytological (ie. morphological) diagnosis of oesophageal brushings did not correlate well with the pathological diagnosis and that cytology alone is not a good enough diagnostic test for oesophageal malignancies.

### Surface Sampling and Techniques

In a preferred embodiment, the invention involves the sampling of the cells from the surface of the oesophagus using a swallowable abrasive material, which material is retrieved from the patient and from which the cells are subsequently separated for analysis.

Preferably substantially the entire surface of the oesophagus is sampled, preferably the entire surface. Prior art techniques focus on sampling from within known or visible areas of Barrett's oesophagus. The present invention advantageously provides for sampling the whole internal surface of the oesophagus ie. the complete inner lumen.

By abrasive is meant that the material is capable of removing cells from the internal surface of the oesophagus. Clearly, since this is meant for use in a subject's oesophagus, ‘abrasive’ must be interpreted in the light of the application. In the context of the present invention the term ‘abrasive’ has the meaning given above, which can be tested by passing the material through the oesophagus in an appropriate amount/configuration and examining it to determine whether cells have been removed from the oesophagus.

The material must be sufficiently abrasive to sample any dysplastic cells present in the oesophagus. Preferably the material is sufficiently abrasive to sample any Barrett's or adenocarcinoma cells present. In a most preferred embodiment, preferably the material is sufficiently abrasive to be capable of sampling the whole oesophagus ie. so that some squamous cells are collected together with any Barrett's and/or columnar and/or adenocarcinoma cells which may be present. This is advantageous because squamous cells are more difficult to remove than dysplastic cells and so their sampling provides a control to the operator such that if normal squamous cells are removed by the material then the chances of having not sampled the cells of interest such as Barrett's or dysplastic cells (if present), which are easier to remove than normal squamous cells, is correspondingly small.

Preferably the swallowable abrasive material is expandable. In this embodiment, preferably the abrasive material is of a smaller size when swallowed than when withdrawn. An expandable material may be simply a resilient material compressed such that when released from compression it will expand again back to a size approximating its uncompressed size. Alternatively it may be a material which expands eg. upon taking up aqueous fluid to a final size exceeding its original size.

In other words, preferably the material of the device expands, swells, inflates or otherwise increases in size between swallowing and withdrawal. Preferably the device is auto-expandable ie. does not require further intervention between swallowing and expansion. Preferably the device is not inflatable. Preferably the device expands by unfolding, unfurling, uncoiling or otherwise growing in size following removal of restraint after swallowing. Preferably the material of the device is compressible and reverts a size approximating its uncompressed size following swallowing. Preferably the device is constructed from a compressed material which is releasably restrained in a compressed state. Preferably the material is released from restraint after swallowing, allowing expansion of the device/material before withdrawal.

Preferably the device comprises compressible material which is compressed into capsule form. Preferably the compressible material is in the form of sponge material.

Preferably the compressed sponge is at least partially surrounded by a soluble and/or digestible coat such as a capsule coat. Preferably the sponge is indigestible. Preferably the capsule coat is at least partially formed from gelatine. Preferably the capsule coat is fully formed from gelatine.

In one embodiment it may be desirable to make the whole device out of digestible material to increase safety in case of a device becoming lost in the subject. Naturally the abrasive material would need to be digested at a slower rate than the capsule and the cord would need to be similarly slowly digested. Preferably the abrasive material is non-digestible. Preferably the cord is non-digestible.

Preferably the abrasive material comprises polyurethane, preferably polyurethane sponge.

Preferably the device is a capsule sponge. As will be apparent from the specification, a capsule sponge is a device comprising compressible sponge as the abrasive material, which sponge is compressed into a capsule shape, which capsule shaped compressed sponge is preferably reversibly restrained in its compressed state by at least a partial coat of soluble and/or digestible material such as gelatine. Preferably the device is a capsule sponge as supplied by Francois Venter at Medical Research Council, South Africa.

Preferably the sample does not comprise endoscopically collected material. Preferably the sample does not comprise endoscopic biopsy. Preferably the sample does not comprise endoscopic brushings.

Preferably the expanded (eg. decompressed) abrasive material of the device is approximately 3 cm in the plane perpendicular to the axis of the oesophagus. Preferably this is the approximate diameter of the oesophageal lumen. More preferably this is slightly larger than the diameter of the oesophageal lumen, advantageously ensuring good contact with the inner surface of same as withdrawal/sampling takes place.

It is a feature of the invention that the sampling is not directed eg. visually directed to any particular part of the oesophagus. It is a further advantage of the invention that a greater proportion of the surface of the oesophagus is sampled than is achieved by prior art techniques such as endoscopic biopsy (which samples approximately 1% of the surface) or endoscopic brushing. Preferably at least 10% of the oesophageal surface is sampled, preferably at least 20%, preferably at least 30%, preferably at least 40%, preferably at least 50%, preferably at least 60%, preferably at least 70%, preferably at least 80%, preferably at least 90%. In a most preferred embodiment, preferably substantially the entire oesophagus is sampled, preferably the whole inner lumen of the oesophagus is sampled. This applies equally to the in vitro sample even when the method of the invention does not include collection of the sample.

### Screening and Surveillance

Screening aspects of the invention relate to the detection and/or diagnosis of Barrett's oesophagus. Typically in screening embodiments of the invention, the subjects being examined, or from which the sample(s) are (or were) obtained, are of unknown status for Barrett's.

Surveillance aspects of the invention relate to the detection and/or diagnosis of dysplasia, including adenocarcinoma Although clearly dysplasia and adenocarcinoma are pathologically different conditions, adenocarcinoma can be regarded as one extreme form of dysplasia. As is discussed below, the invention may be advantageously applied to distinguish adenocarcinoma from dysplasia, depending upon the molecular markers used. However, in general the discussion of surveillance aspects of the invention relates to the detection of dysplasia, including adenocarcinoma. Typically in surveillance embodiments of the invention, the subjects being examined, or from which the sample(s) are obtained, are of unknown status for dysplasia but will typically be known to have Barrett's.

In principle the difference between screening and surveillance aspects is of little practical consequence to the working of the invention. The difference relates only to the markers chosen. The sampling and combination aspects remain the same between screening and surveillance. Indeed, it may be advantageous to combine screening and surveillance ie. to examine cell samples for markers of Barrett's as well as dysplasia including adenocarcinoma at the same time, thereby increasing the value of the information obtained and achieving a more robust combined diagnostic output.

### Markers

Markers that can be applied for Barrett's screening and surveillance are any markers which are not expressed in normal oesophageal tissue, preferably any markers which are not expressed in normal oesophagal surface cells. Preferably markers are markers of non-squamous cells. Preferably markers are markers of cellular proliferation.

For screening aspects (ie. for detection of Barrett's oesophagus), preferably markers that distinguish between intestinal metaplasia (Barrett's) and squamous oesophageal cells or gastric cardia are used. These markers include markers of epithelial differentiation.

### Screening—Columnar Markers

Preferably the marker is a marker of columnar cells.

Preferably such markers include brush border proteins such as villin or moesin, brush border enzymes such as alkaline phosphatase, which are expressed specifically in specialised intestinal metaplasia. Homeobox genes such as Cdx1 and/or Cdx2 are further examples of such useful markers, in that columnar tissue but not squamous express homeobox genes such as CDX-1, CDX-2.

Furthermore, specific mucins are expressed in Barrett's but not in gastric tissue (e.g. MUC2A, MUC2B).

Other types of columnar metaplasia and native columnar tissue can be differentiated from squamous epithelium according to their cytokeratin expression profile (e.g. CK 7, 8, 13, 14, 18). In particular, cytokeratins such as CK7 and/or CK8/18 for columnar cells versus CK13/14 for squamous cells are useful markers according to the present invention.

The use of columnar markers is particularly preferred. The technical benefit of using columnar markers is that only columnar cells are detected by using them. This means that squamous cells (whether normal or cancerous) are not stained by columnar markers. This is an advantage because Barrett's cells and dysplastic cells arising therefrom such as adenocarcinoma cells are columnar and can thus be selectively identified by use of columnar marker(s). This advantageously improves signal and also reduces background and alleviates the need to apply further distinguishing markers, thereby simplifying the procedure by directly detecting columnar cells in this manner.

Particularly preferred are the columnar markers mentioned above, preferably columnar markers such as brush border proteins and/or homeobox genes and/or mucins and/or cytokeratins.

Preferably combination aspects of the present invention, such as kits and methods, include at least one columnar marker.

### Screening—Lectin Markers

Lectins are very abundant proteins. Lectins/lectin binding partners are expressed more in BE than in normal tissue. Lectins are glycoproteins which selectively bind to specific configurations of carbohydrates such as mucins expressed in BE. Cell-surface molecules, including growth factor receptors are frequently glycosylated, and lectins may also bind to these. When labelled with appropriate fluorochromes lectins can be highly sensitive, quantifiable and specific tools for detection and prognosis of dysplastic and invasive cells using established histochemical and flow cytometry protocols (e.g. Jordinson M 1998). Their low cost, high abundance and affinity, through multiple binding sites, make them very attractive as biomarkers. We have generated data to demonstrate that three preferred fluorochrome-bound lectins (Helix pamatia agglutinin (HPA), peanut agglutinin lectin (PNA) and Ulex europaeua agglutinin-1 (UEA-1)) can discriminate between non-dysplastic and dysplastic cell lines and tissues. The fluorochrome is highly stable and is amenable to automated microscopic analysis or quantitative assessment by flow cytometry. Thus, lectins are preferred markers of the invention.

### Screening—General Markers

Markers from pathways regulating cellular differentiation are also usable to distinguish cells in screening embodiments, in particular the wnt pathway and Notch pathway genes.

Any other markers known to be differentially expressed in Barrett's versus normal oesophageal surface cells may be employed.

Alternative markers may be identified using an expression microarray comparing gastric cardia and squamous cell biopsies. Any marker which is differentially present in these cell types may be used in the present invention.

Preferred markers for detection of Barrett's oesophagus (ie. for use in screening embodiments of the invention) are villin or moesin, preferably villin.

### Surveillance

For surveillance aspects, preferably markers whose expression correlates with the degree of dysplasia are used. Preferably such markers are used for the stratification of patients at risk.

Preferably such markers include proliferation markers such as Ki67 and Mcm proteins, proliferation and DNA damage markers such as PCNA, cyclins such as cyclin D and/or cyclin A, aberrant p53 for example p53 LOH, p53 mutation, or p53 overexpression such as immunohistochemical detection thereof, p16 loss including methylation, and aneuploidy for example measured by flow cytometry or image cytometry.

In slightly more detail, growth factors (such as EGF), growth factor receptors (such as EGFR) as well as cytokines (IL-4) and molecules involved in inflammatory response (COX-2) were shown to have an aberrant expression in BE and subsequent progression to AC, and are therefore useful markers according to the present invention.

Progression to adenocarcinoma is likely to lead to increased proliferation. Proliferation markers (e.g. MCM proteins, Ki-67, PCNA) are considered to be markers of progression. Markers expressed during the cell cycle, therefore tightly linked to proliferation are also markers of use herein (e.g. cyclin, pRb). Markers exerting a negative control on the cell cycle are of interest such as cycle inhibitors, like CDK inhibitors (p15, p16).

In vitro and ex vivo work has shown that acid and bile stimulation induced DNA damage, MAP kinase pathway and the NFκB pathway and decreased apoptosis therefore markers involved in the detection of DNA mutation and damage (e.g. ATM, ATR), markers of apoptosis (p53) and markers from the MAPK pathway (erk, p38) and markers from the NFκB are useful. Furthermore, bile acids increase the retinoic acid pathway (CYP26A1, RAR) which is linked to the induction of metaplasia in chick embryo oesophagus. A number of other pathways have been involved in the development of BE and progression to cancer such as TGFβ and BMP pathways.

### Markers: Further Considerations

Indeed, any marker known to correlate with the degree of dysplasia would be suitable, including many oncogenes and tumour suppressor genes. In particular, markers mentioned in Fitzgerald RC Clin Gastroenterol Hepatol Complex diseases in gastroenterology and hepatology: GERD, Barrett's, and esophageal adenocarcinoma. 2005, 3:529-37 or in Fitzgerald RC Recent Results in Cancer Res Genetics and prevention of oesophageal adenocarcinoma 2005, 166:35-46 may be suitable for use in the present invention.

Mcm markers and/or cyclin A are particularly preferred for detection of Barrett's associated dysplasia including adenocarcinoma, most preferred are Mcm markers. Preferred Mcm (minichromosome maintenance) markers are one or more of Cdc6, Mcm2, Mcm3, Mcm4, Mcm5, Mcm6, Mcm7 or Mcm8, preferably Mcm2. When the marker is Mcm2 a sensitivity of 85% and a specificity of 70% or even more is achieved.

If using an Mcm marker alone, then detection of mere Barrett's oesophagus is only likely to be made in a proportion of cases, ie. many early stage Barrett's lesions will not show Mcm expression. Thus, if applying the invention to simultaneous screening and surveillance, preferably a separate screening (ie. Barrett's oesophagus) marker is advantageously selected to be used in combination with the surveillance marker Mcm.

In a highly preferred embodiment a single marker Mcm2 or Cyclin A is used. Preferably Mcm2 is used. Mcm2 detects approximately half of all incidences of Barrett's oesophagus together with Mcm2 positive dysplasias and cancers. The technical advantage of this embodiment is that, although up to half of the occurrences of Barrett's may not be detected, these are the early stage Barrett's and the ones which are detected by Mcm2 alone are the highest risk group of Barrett's. Thus, by using a single Mcm2 marker the procedures are simplified and the maximally important group of disorders is reliably detected. Preferably when using Mcm2 alone, the cells are collected by capsule sponge. The NPV (negative predictive value) for Mcm2 in the detection of cancers and high grade dysplasia is 100%; therefore a patient negative for Mcm2 will not have HGD or AC. The PPV of 72% for the detection of cancer and dysplasia compared with non-dysplastic BE means that 72% of patients positive for Mcm2 will be dysplastic. Furthermore, 90% of patients with Mcm2 positivity will have an oesophageal abnormality. (Of course these figures and those below are based on a population study and should be interpreted accordingly; for more detail see the examples section.)

Cyclin A is disclosed to be indicative of Barrett's for the first time herein. Thus, preferably the marker is Cyclin A. Cyclin A alone has a sensitivity of approximately 95% and a specificity of approximately 65% (positive (PPV) 58%, negative (NPV) 98%). Furthermore, Cyclin A levels increase during progression from Barrett's to low grade dysplasia to high grade dysplasia to adenocarcinoma. Thus in one embodiment the invention relates to quantification of Cyclin A, preferably on a per cell basis, and correlation with likely state of abnormality.

Preferably the marker used is Cyclin A. More preferably a combination of Cyclin A with one or more other marker(s) disclosed herein is used.

Cyclin A is more specific than Mcm2 but fractionally less sensitive. In a preferred embodiment, both Mcm2 and Cyclin A are used in combination. These markers have a negative predictive value of near 100% and in combination have a positive predictive value for dysplasia and cancer of around 50%. Thus, if a subject was negative for Mcm2 and Cyclin A then this is indicative of lack of Barrett's associated dysplasia including adenocarcinoma.

It should be noted that the present invention is not concerned with the diagnosis of squamous cell carcinoma of the oesophagus. This is a quite different disorder to Barrett's oesophagus and to Barrett's associated dysplasias such as adenocarcinoma. Preferably the diagnosis of squamous cell carcinoma of the oesophagus is specifically disclaimed from the present invention.

### Marker Assay/Detection

Assaying for a marker means determining the presence or absence of said marker. Preferably assaying means immunological staining or visualisation of the marker.

Marker expression (marker gene expression) may be detected by any suitable means known to those skilled in the art. Expression may be detected at the nucleic acid or protein level. Expression may be by mass spectrometry and assignment of the mass readouts to particular protein moieties. At the nucleic acid level, detection is preferably by monitoring of mRNA levels. Preferably expression is detected at the protein level. Preferably marker gene expression refers to marker protein expression. Preferably marker protein expression is determined by direct or indirect detection of marker protein. Preferably such protein is detected by immunochemical means. Preferably the marker protein is detected by an antibody capable of reacting with that protein, and subsequent visualisation of said antibody. Preferably the antibody is a polyclonal antibody or a monoclonal antibody. Preferably when the antibody is a polyclonal antibody it is an immunopurified polyclonal antibody. Preferably the antibody is a monoclonal antibody. Use of secondary and even tertiary or further antibodies may advantageously be employed in order to amplify the signal and facilitate detection. Preferably marker protein(s) are visualised by use of immunohistochemical means, such as immunofluorescent means, directly or indirectly bound to the marker protein(s). Preferably detection is by antibody to the marker.

Other suitable assays include ELISA—fluorescent in-situ hybridisation of fish and FACS—fluorescence analysis of cell sorting.

### Sample

It will be appreciated that the sample preferably comprises a population of individual cells obtained by the sampling procedures described herein. Thus, the detection of the markers preferably refers to detection of the markers in at least one cell within said population of cells. The detection of a proliferative marker in any cells in the sample will be indicative of Barrett's or a Barrett's associated dysplasia. The absence of any cells showing the marker from the population of cells of the sample will be indicative of lack of Barrett's or Barrett's associated dysplasia. The proportion of cells showing expression of the marker is less important. The proportion of cells showing expression of the marker would not usually make a contribution to the diagnosis. The present invention is based on the detection of any cell(s) showing the marker in the sampled cell population, or the apparent absence of any cells showing the marker. In some embodiments, it may be advantageous to determine the relative proportions of the cell types or the proportion of cells displaying proliferative markers, as an optional step dependent on the needs of the operator. However, for most embodiments of the invention, the result will be expressed as a positive or negative, and the relative proportions of cells will normally not be taken into consideration.

### Kits

The kits of the invention are designed to provide for conducting the methods of the present invention. Thus, the description of elements required for the methods of the invention applies equally to the contents of the kits of the invention, which preferably contain the elements required for practice of said methods. In particular, preferably the kits contain reagent for detection of the marker or markers being used.

Preferably the kit of the invention also contains a local anaesthetic for use in the oesophagus. Preferably this may be in the form of a spray or lozenge, preferably a spray.

Preferably the kit of the invention also contains a container for holding the device once withdrawn from the subject. Preferably this container is watertight. Preferably the container contains a preservative fluid. Preferably the container contains a liquid based cytology fluid such as commercial thin preparation fluid for producing slides of the sampled cells. Preferably the thin preparation fluid comprises a preservative.

Preferably the swallowable device is lubricated to aid swallowing, preferably the withdrawal means is also lubricated. Thus, preferably the kit comprises lubricant.

Preferably the kit comprises a drinkable solution to aid swallowing the device. Preferably said solution is flavoured to disguise the taste of the device, or to render it more palatable. Preferably said solution is thickened eg. by addition of sugar or pectin or other agent giving rheological characteristics such as viscosity or thickness. The advantage of this is that a more viscous or dense solution will be more effective at aiding passage of the device through the oesophagus during swallowing.

In order to save weight/volume in kits, preferably the solution(s) supplied are supplied in powdered form such that the operator reconstitutes them before use eg. by adding water. Preferably the kit comprises a container for reconstitution. Preferably said container is graduated to facilitate measurement of the correct amount of fluid such as water.

Preferably the swallowable device does not comprise animal product(s).

Preferably the kit comprises anti-emetic eg. in lozenge, solution or powdered form, to suppress any urge to vomit during introduction and/or withdrawal of the device.

Preferably the kit may comprise antacid such as acid-neutralising compound(s), or such as pharmaceutical antacid for inhibition of acid production/secretion in the stomach. Advantageously this may be used to inhibit a burning sensation of acid carried up the oesophagus from the stomach upon withdrawal of the device. Furthermore, this may be advantageous in preservation of the cell samples obtained with said device.

Preferably the preservative fluid contains antacid and/or is buffered to the desired pH for preservation of the cell sample obtained.

In one embodiment the kit preferably comprises a local anaesthetic spray, a capsule sponge, a pot containing prep liquid (e.g. ThinPrep™ PreservCyt™ Solution™), a label for the pot, and an instruction leaflet for a health care professional who administers the sampling.

Preferably the kit further comprises gloves (for health care professional such as a nurse removing the capsule from the subject).

Preferably the kit further comprises scissors to cut the withdrawal means (e.g. string).

Preferably the kit further comprises a plastic cup (for subject to drink fluid e.g. water).

Preferably the kit further comprises an information leaflet for the subject/patient.

In another embodiment the invention relates to a self testing kit such as a dip-stick format kit whereby said stick comprises reagents for detection of markers according to the present invention and wherein in use dipping the stick into the pool of sampled cell material leads to a visualised readout of the markers according to the present invention, thereby providing information capable of aiding diagnosis as set out herein.

Preferably the device comprises integral withdrawal means. Preferably this is a string or cord based means. Preferably the withdrawal means is graduated so that the operator can estimate when the device is, or is likely to be, in the stomach. Furthermore, the graduations advantageously allow monitoring of withdrawal of the device and allow for standardisation of the rate of withdrawal and for optimisation of sample collection.

Preferably the withdrawal means comprises an unswallowable element at the end distal from the swallowable abrasive material. This advantageously prevents accidental swallowing of the entire device, inhibiting or preventing its withdrawal. Preferably this unswallowable element is detachable in case of emergency when it may be safer to allow the entire device to be swallowed and passed through the alimentary canal.

### Further Kit Features

In some embodiments, it is probable that there will be a multi-part kit to provide for different elements in different settings. The discussion above is focussed on the preferred aspects of the kit of the invention which is the primary care application e.g. in screening for initial detection in a subject. However, it will be apparent to the skilled person that the oesophagus surface sample may be analysed at a location different from the initial primary care setting in which subject(s) are sampled. For example, the cell(s) may be analysed in a laboratory separate from the primary care setting in which the sample is collected. In this embodiment it is apparent that the invention may relate to multi-part kit(s) having a primary care component as well as a read-out component (or laboratory component), or the invention may even relate to the read-out/laboratory component of the kit per se. In this example, the read-out (or laboratory) component of the kit may comprise one or more of the following elements:
Consumables such as non-gynaecological microscope slides, and/or non-gynaecological filters.
Equipment such as ThinPrep™ 2000 processor.
Detection of abnormal pathology—for the detection of Barrett's oesophagus using immunohistochemistry for Mcm2; System for automated immunostaining e.g. if the samples are stained using the DakoCyomation Ltd ChemMate™ system.

The kit may further comprise one or more of the following detection consumables such as Dako Autostainer reagents vial; ChemMate™ detection kit; ChemMate™ Peroxidase blocking solution; ChemMate™ antibody diluent; Mcm2 antibody; Goat serum; Bovine serum albumin; Haematoxylin and/or Coverslips.

The kit may further comprise equipment such as Dako autostainer slides processor (S3400 Dako autostainer).

In order to facilitate analysis of the samples, the kit may comprise visualisation means such as a microscope (such as an automated microscope) e.g. Olympus BX41 with X10, X20 and/or X40 objectives.

### Further Advantages/Applications

Once tissue architecture is lost as in surface sampling, cytologists can no longer tell cell types such as squamous, columnar, Barrett's etc apart. Furthermore, observation of inflammatory cells such as lymphocytes no longer contributes to the diagnosis since no positional information can be gleaned from their observation. However, advantageously the present invention overcomes this problem by employing biomarkers to identify the cell types even when the histological information has been lost.

Analysis of cells for marker expression is often performed by distributing the cells on microscope slides followed by staining and analysis. By visualising markers in the cells, the present invention advantageously allows automation since judgement of a histologist is no longer required based on the cell architecture, but rather a positive/negative signal for presence/absence of the marker is the readout. This readout can be quickly collected by image capture, and data analysis/diagnosis can advantageously be uncoupled from staining/imaging steps of the procedure. Furthermore, preferred sampling devices of the present invention such as capsule sponges advantageously collect more cells than laborious prior art techniques such as endoscopic brushings. Specifically, approximately 6-12 times more cells can be collected in a single capsule sponge sample than in a hazardous endoscopic brushing.

A key difference over the prior art is the collection of only surface samples. Rather than being a disadvantage as would be expected from the art, this is in fact an advantage of the present invention in that for example any surface cell showing proliferative marker such as Mcm2 or Cyclin A is abnormal, which might not be true for cells sampled from deeper in the oesophagus where active division might be taking place with no implication of potentially pathologic condition. Thus, it is an advantage of the present invention that surface-only cells are assayed.

Capsule sponges have been applied in detection of squamous cell carcinoma. It is an unexpected advantage of the present invention that these capsule sponges can be applied to the detection of the quite different Barrett's associated disorders.

Conventional Barrett's sampling techniques such as the gold standard biopsy at best sample approx. 1% of the surface area of the oesophagus. The present invention advantageously samples approximately the entire surface area of the oesophagus.

Although it is preferred to assay the cells by distribution onto slides, it may be advantageous to perform the assay in a different format such as ELISA or FACS or FISH. Preferably the cells can be assayed in one or more of these format(s) directly from the capsule sponge or washings therefrom, advantageously avoiding the need for a slide format analysis. If a slide format analysis is required, preferably cells are concentrated onto the slides to produce fewer slides for the same number of cells, thereby saving costs. In one embodiment, preferably the cells from the capsule sponge are collected and their protein extracted and tested for the marker(s), thereby alleviating the need for whole cell staining.

Advantageously pore size on the preferred capsule sponge sampling device can be varied to regulate the number of cells harvested. For example, by reducing pore size the number of cells (and thus the number of slides needed) may be advantageously reduced.

In highly preferred embodiments, markers are chosen to detect high risk Barrett's. This has the further advantage that surveillance ie. remonitoring of patients with Barrett's to detect future dysplasia including adenocarcinoma may be reduced or rendered unnecessary since in one step the Barrett's is detected and graded as high risk, so subsequent treatment can be prescribed immediately without expensive surveillance, and without the risk that during surveillance the patient will go on to develop more dangerous lesions before detection.

Advantageously the techniques of the present invention are applicable in primary care ie. in general practitioners' surgeries where the samples can be taken and processed remotely in batch form, thereby reducing costs and reducing patient time lost. Furthermore, the techniques can be carried out by staff at general practitioners' surgeries, advantageously avoiding the need for specially trained personnel such as doctors to be involved in the sampling/processing.

It is an advantage of the present invention that false negatives are extremely rare. Some false positives can occur, eg. detection of naturally proliferating cells such as closing a wound incurred by swallowing an abrasive foodstuff such as a fruit stone. However, a negative result from the tests and kits of the present invention is very reliable so that patients can be excluded from unnecessary follow up procedures and can receive robust reassurance at an early stage when a negative result is obtained. Since the methods and kits of the invention are simple and low in cost, a much wider screening programme can be undertaken for the same net cost to the service provider.

Preferably the tests of the present invention are carried out on a given subject at 3 year intervals.

A further advantage of the invention is that liquid based cytology is possible, which is superior to conventional cytology employed in Barrett's in the prior art.

Another advantage of the invention is that the first signs of dysplasia can be very small and may be missed by visual inspection or endoscopic biopsy sampling, but will be detected according to the present invention. Similarly, 40% of subjects with high grade dysplasia already have the cancer present. The present invention advantageously allows better detection/diagnosis of these patients.

A further advantage of the present invention is that it does not require endoscopy and is therefore cheaper and easier and safer than prior art techniques. Thus, according to the present invention the oesophagus is not sampled by endoscopy. In particular, it is a key feature of the present invention that the surface of the oesophagus is sampled. Endoscopic biopsies typically sample a depth of tissue rather than merely the surface. It is a surprising advantage of the invention that surface-only sampling can be used to aid the diagnosis of Barrett's oesophagus or Barrett's associated dysplasia. Preferably the oesophagus surface is sampled non-endoscopically. This has the further advantage(s) of being quicker, cheaper, and is suitable for population screening in primary care.

Preferably the invention samples a large surface area of the oesophagus. Endoscopic sampling only samples a small surface area of the oesophagus. Sampling a large surface area has the advantage of decreasing the chances of missing an abnormality due to limitation of the coverage at the point of sampling.

Preferably the invention relates to screening (e.g. population screening) applications i.e. detection of initial abnormality. Preferably the invention is suitable for population screening in primary care.

Diagnosis according to the present invention is advantageously more consistent, eliminating operator variation with prior art techniques such as brushing/biopsy.

The invention preferably does not involve sampling techniques based on devices featuring rigid stems or cables. These are difficult or impossible to swallow. Preferably the capsule sponge as described above is used in the methods and kits of the invention. This has the advantage of being easily swallowed. Furthermore, it has the advantage of being able to collect cells throughout its structure due to its preferred mesh construction, rather than being limited to collection on the cell surface as is the case with prior art devices. This has the advantage of increased yields.

Surprisingly, a non-directional sampling method of the invention does not suffer from overwhelming background of squamous cells which would be expected from an understanding of the prior art. This is because prior art techniques are directed at the Barrett's which typically makes up only 2-5% of the surface area of the oesophagus or even less, whereas the present invention samples the whole surface area so that it would be expected that any Barrett's signal would be masked by background but it is surprisingly shown herein that this is not the case.

The present invention will now be described, by way of example only, in which reference will be made to the following figures:

### BRIEF DESCRIPTION OF THE FIGURES

FIG. 1, which shows a photo of a preferred sampling device of the invention before swallowing.

FIG. 2, which shows a photo of a preferred sampling device of the invention, partially disassembled to show the capsule construction. Arrowed is the part of the capsule through which the cord passes.

FIG. 3, which shows a photo of a preferred sampling device of the invention, partially disassembled to show the capsule construction.

FIG. 4, which shows a photo of a preferred sampling device of the invention after dissolution of the capsule and expansion of the expandable material.

FIG. 5, which shows an expanded capsule sponge.

FIG. 6, which shows photographs of stained cells.

### EXAMPLES

### Example 1

### Construction of a Sampling Device

Abrasive material is cut to the appropriate size. In this example, the material is approximately the size of the internal diameter of a human oesophagus, ie. approximately 3 cm in diameter.

In this example the material is a polyurethane mesh or cloth.

A cord is stitched into the material so that it can be retrieved after swallowing. (FIG. 3 shows the device with cord attached).

The cord is sufficiently long that part of it will comfortably remain outside the buccal cavity even after the device has been swallowed and resides in the stomach. The cord and stitching is sufficiently strong and resistant to digestion so that it can be used to retrieve the device after expansion.

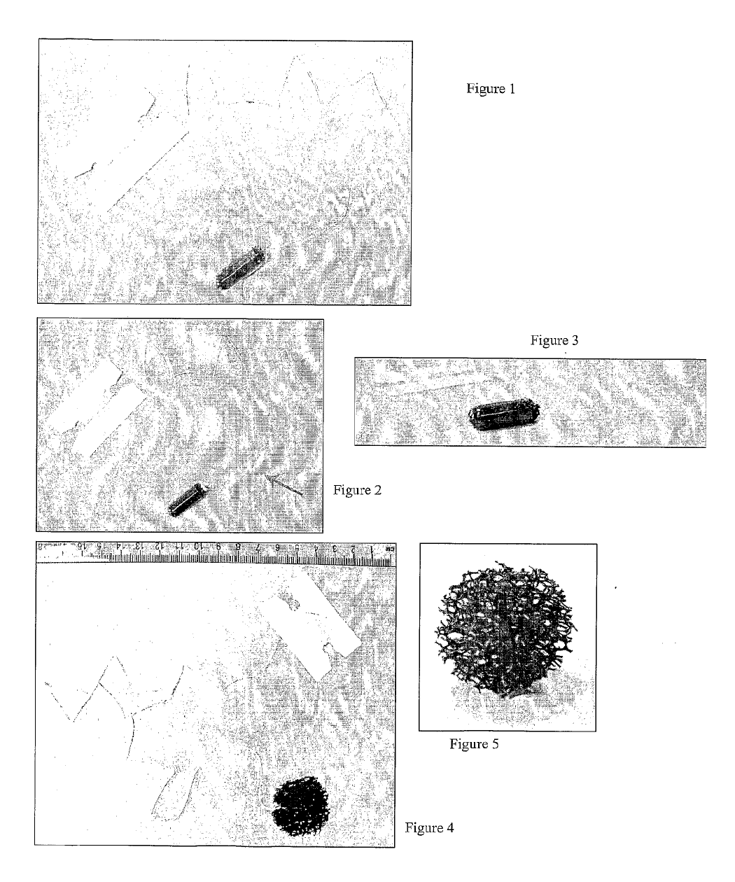

The material is then compressed and inserted into a gelatine capsule (FIG. 1). The cord exits the capsule (FIG. 2 shows partially disassembled capsule with cord exiting). The device is then ready for use.

### Example 2

### Sampling of Cells from the Surface of the Oesophagus

A device according to example 1 is provided. The subject may tale a local anaesthetic in the form of a lozenge or spray by way of preparation.

The device is introduced into the subject's buccal cavity with the distal end of the cord retained outside the buccal cavity.

The device is then swallowed. A drink of warm water aids this process and wets the cord, facilitating its passage down the oesophagus.

After approximately 10-20 seconds the device arrives in the subject's stomach, the cord exiting the stomach and lying in the oesophagus and the buccal cavity and outside to the point of retention.

After 5 minutes the capsule coat has dissolved and the abrasive polyurethane material has expanded back to its uncompressed size.

The device is then withdrawn by gentle tension on the distal end of the cord, pulling the device along the oesophagus and out of the buccal cavity, collecting oesophageal cells en route. The device is then stored in a preservative fluid in a sealed container until processing for assay of the sampled cells. Preferably the preservative fluid is thin preparation fluid for production of analytical slides.

### Example 3

### Assaying for Cellular Markers

The withdrawn device of example 2 is washed to collect the oesophageal cells. These are then applied to slides and fixed for visualisation.

Mcm2 is the marker in this example.

The numbers analysed for this part of the study are 18 BE patients and 22 healthy controls). The age of the BE patients was 64.5±2.1 years compared to 31.2±1.6 for the healthy volunteers and the male:female ratio was biased towards a male population in both groups (1:5 and 1:1.7 respectively).

The PAP slides were used to assess the cellularity of the samples. An expert cytopathologist assessed the cellularity of the samples and 88% of the samples had a good to very good cellularity and 22% had an average cellularity.

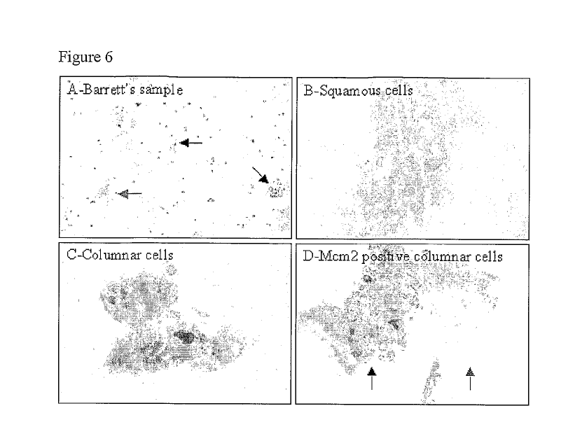

FIG. 6 shows Representative pictures of monolayers from capsule sponge samples. Pap stained samples (A-C) and Mcm2 stained sample (C). The black arrows indicate the position of columnar cells and the red arrows the position of squamous cells.

As seen with endoscopic brushings, columnar and squamous cells were easily distinguishable (FIGS. 6A, 6B and 6C) on PAP stained samples. Mcm2 positive cells were stained as strongly as those seen from endoscopic brushes (FIG. 6C).

This non-endoscopic technique has at least two industrial applications. The first is to identify all of the Barrett's patients ie. to demonstrate use of the invention as a screening test to detect Barrett's oesophagus. The second is to stratify the BE patients according to their risk of progression to adenocarcinoma (ie. to demonstrate use of the invention in surveillance). The two interdependent applications can be achieved by altering the biomarkers used.

Columnar cells were detected in 61% of BE patients and in 9% of control patients (table A).

TABLE A

| | Columnar cells |
|---|---|
| Barrett's | 11/18 (61%) |
| Control | 2/22 (9%) |
| Sensitivity | 61% |
| Specificity | 91% |
| PPV | 84% |
| NPV | 74% |
| Efficiency of the test | 77% |

Table A shows Mcm2 positivity and presence of columnar cells in capsule sponge cell samples from Barrett's patients (with or without dysplasia) and control patients. The results of analysis are in the bottom panel (PPV: positive predictive value and NPV: positive negative value).

As discussed, we have shown that surface expression of Mcm2 is associated with a higher risk for cancer progression. Mcm2 expression was detected in 55% of BE samples and 9.1% of NE samples (table B).

TABLE B

| | Mcm2 positivity |
|---|---|
| NE | 2/22 (9%) |
| BE | 5/9 (55%) |
| LGD | 4/8 (50%) |
| HGD | 1/1 (100%) |

Table B shows Mcm2 stained capsule sponge samples in the diagnosis of BE and associated dysplasia. The values for the positive brushings represent the number of patients with any discernable Mcm2 expression. (NE: normal oesophagus, BE: Barrett's oesophagus, LGD: low grade dysplasia, HGD: high grade dysplasia).

The percentage of samples detected by Mcm2 staining correlated with increasing degree of dysplasia (p<0.05).

TABLE C

| Length of segment (cm) | Mcm2 positivity | Columnar cells |
|---|---|---|
| Short | 0/1 | 0/1 |
| 2 | 1/2 | 1/2 |
| 3 | 1/2 | 1/2 |
| 5 | 1/4 | 2/4 |
| 8 | 1/3 | 3/3 |
| 10 | 3/3 | 3/3 |

Table C: Mcm2 positivity and presence of columnar cells in capsule sponge in relation to the length of the Barrett's segment.

There is a correlation between the length of the segment and the presence of columnar cells (table 4-9, p<0.05) but no correlation with Mcm2 positivity.

Thus the value of assaying surface cell samples for biomarkers in order to aid the diagnosis of Barrett's and Barrett's associated dysplasia such as adenocarcinoma is demonstrated.

### Example 4

### Development and Evaluation of a Non-Endoscopic Immunocytological Screening Test for Barrett's Esophagus

Background: Barrett's esophagus (BE) is an established risk factor for oesophageal adenocarcinoma; however, the majority of patients are undiagnosed. Endoscopic population screening for BE is impractical and wireless capsule imaging devices do not permit tissue sampling. Previous non-endoscopic cytological sampling devices have been poorly tolerated and cytological analysis is inadequate for the accurate assessment of BE.

In this example we demonstrate a method for aiding the diagnosis of Barrett's oesophagus or Barrett's associated dysplasia in a subject. The method comprises sampling the cellular surface of the oesophagus of said subject, wherein said sampling is not directed to a particular site within the oesophagus. In this example, sampling is by means of capsule sponge.

The method then involves assaying the cells for a non-squamous cellular marker. In this example, the marker is Mcm 2. We show that immunocytological assessment of the proliferation marker minichromosome maintenance protein 2 (Mcm2), is a useful method for detection and monitoring of BE since proliferation is progressively dysregulated from early in the disease pathogenesis. This example demonstrates a non-endoscopic screening test for BE which uses a capsule-sponge device in combination with Mcm2 staining.

In this technique, detection of such a Mcm2 marker indicates increased likelihood of the presence of Barrett's or Barrett's associated dysplasia.

Methods: Following routine optimisation of the preferred capsule sponge device in combination with immunocytology, 27 BE patients (with endoscopically visible glandular mucosa containing intestinal metaplasia on biopsy) and 30 normal healthy volunteers were recruited to the study.

Patients swallowed the sponge and 5 minutes later the expanded sponge was placed into preservative. Liquid based cytology was used to create a cell-monolayer in which the maximum number of cells possible was extracted from the device. Immunocytochemistry was performed with a mouse monoclonal antibody against Mcm2. A binary score was generated such that a single cell with nuclear Mcm2 positivity led to a positive score being assigned.

Two individuals unaware of the clinical diagnosis assessed the slides.

To determine the acceptability of the test, the patients used a linear rating tool.

Results: Inadequate specimens were retrieved from 3/57 (5.2%) patients. None of the squamous cells retrieved from any patient had Mcm2 positivity. 22/26 (84%) patients with BE had columnar Mcm2 positivity compared with 7/28 (25%) healthy volunteers giving a sensitivity and specificity of 84.6% and 75% respectively. The negative and positive predictive values of the test are 75.9% and 84.0% respectively.

The acceptability of the capsule was rated as 4.4+/−0.3 with 10 being very enjoyable, 5 being neither unpleasant nor pleasant and 0 very unpleasant.

Conclusions: The sensitivity and specificity of capsule-sponge immunocytology demonstrated compares favourably with other screening tests in current clinical practice.

Thus it is demonstrated that non-endoscopic immunocytology according to the present invention is applicable to primary care, and that automated processing can be used. Thus, methods of the invention represent useful screening tools for BE.

### Example 5

### Development and Evaluation of a Non-Endoscopic Immunocytological Screening Test for Barrett's Oesophagus

Background: Barrett's oesophagus (BE) is a risk factor for oesophageal adenocarcinoma; however, the majority of patients are undiagnosed. The aim of this study was to develop a non-endoscopic screening test for BE and to show it is suitable for application in a primary care setting.

This example sets out a method for aiding the diagnosis of Barrett's oesophagus or Barrett's associated dysplasia comprising assaying cells from the surface of a subject's oesophagus for a non-squamous cellular marker. In this example, the cells are collected by means of a capsule sponge.

We have previously shown that the surface epithelium of BE contains proliferating cells detectable by immunocytochemistry for Minichromosome maintenance protein-2 (Mcm2). This is the marker used in this example.

We demonstrate that detection of such a Mcm2 marker indicates increased likelihood of the presence of Barrett's or Barrett's associated dysplasia.

Methods: 43 BE patients and 42 healthy volunteers swallowed a capsule-sponge attached to a string. 5 minutes later the expanded sponge was retrieved and placed into preservative. Liquid based cytology was used to create a cell-monolayer which was stained for Mcm2. Samples were considered positive if columnar cells had nuclear staining. Three individuals unaware of the clinical diagnosis assessed the slides. To determine the acceptability of the test, the patients used a linear rating tool (10 enjoyable, 5 neither unpleasant nor pleasant, 0 very unpleasant).

Results: Inadequate specimens were retrieved from 4/83 (4.8%) patients. 27/41 (66%) BE specimens were positive compared with 8/40 (20%) specimens from healthy volunteers giving a sensitivity and specificity of 67% and 80% respectively. The negative and positive predictive values of the test are 77.1% and 71.0% respectively. The acceptability of the capsule was rated as 4.4+/−0.3

Conclusions: The sensitivity and specificity of capsule-sponge immunocytology compares favourably with other screening tests in current clinical practice. Furthermore, the method is applicable to primary care and automated processing could be used in practising the method. This is a useful screening tool for BE. The method may be varied by the use of alternative molecular markers, for example a lectin marker.

We have demonstrated that three fluorochrome-bound lectins (Helix pamatia agglutinin (HPA), peanut agglutinin lectin (PNA) and Ulex europaeua agglutinin-1 (UEA-1)) can discriminate between non-dysplastic and dysplastic cell lines and tissues. The fluorochrome is highly stable and is amenable to automated microscopic analysis or quantitative assessment by flow cytometry. Thus, the utility of lectin markers is also demonstrated herein.

All publications mentioned in the above specification are herein incorporated by reference. Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A kit comprising a swallowable device comprising abrasive material capable of collecting cells from the surface of the oesophagus, together with printed instructions for its use in detection of Barrett's oesophagus or Barrett's associated dysplasia.

2. The kit according to claim 1 further comprising a local anaesthetic.

3. The kit according to claim 1 or claim 2 further comprising a container for receiving said swallowable device after withdrawal, said container having a quantity of preservative fluid therein.

4. The kit according to claim 1, wherein said device comprises a capsule sponge.

5. The kit according to claim 1, wherein said device comprises withdrawal means.

6. The kit according to claim 5 further comprising a device for severing said withdrawal means.

7. The kit according to claim 1, further comprising a container for administering drinkable fluid to the subject.

8. The kit according to claim 1, further comprising gloves.

9. The kit according to claim 1, further comprising reagent for use in the detection of a non-squamous cellular marker.

10. The kit according to claim 9, wherein said non-squamous cellular marker is a marker of cellular proliferation.

11. The kit according to claim 9, wherein the non-squamous cellular marker is a marker of columnar cells.

12. The kit according to claim 9, wherein said kit further comprises reagents for use in the detection of at least one marker selected from the group consisting of brush border proteins such as villin or moesin, mucin genes, brush border enzymes such as alkaline phosphatase, homeobox genes such as Cdx1 and/or Cdx2, cytokeratins such as CK8/18 for columnar cells, or any marker known to be differentially expressed in Barrett's versus normal oesophageal surface cells.

13. The kit according to claim 9, wherein said kit further comprises reagents for use in the detection of at least one marker selected from the group consisting of proliferation markers such as Ki67 and Mcm proteins, proliferation and DNA damage markers such as PCNA, cyclins such as cyclin D and/or cyclin A, abnormal p53, loss of p16, aneuploidy or any marker known to colTelate with the degree of dysplasia.

14. The kit according to claim 1 wherein said marker is Cyclin A.

15. The kit according to claim 9 wherein said marker is a lectin.

16. The kit according to claim 3 wherein said fluid is thin preparation fluid for production of slides for examination of the sampled cells.

17. The kit according to claim 2, further wherein said local anaesthetic is a spray or lozenge.

18. A method for aiding the diagnosis of Barrett's oesophagus or Barrett's associated dysplasia in a subject, said method comprising sampling the cellular surface of the oesophagus of said subject, wherein said sampling is not directed to a particular site within the oesophagus, and assaying the cells for a non-squamous cellular marker, wherein detection of such a marker indicates increased likelihood of the presence of Barrett's or Barrett's associated dysplasia.

19. A method for aiding the diagnosis of Barrett's oesophagus or Barrett's associated dysplasia comprising assaying cells from the surface of a subject's oesophagus for a non-squamous cellular marker, wherein detection of such a marker indicates increased likelihood of the presence of Barrett's or Barrett's associated dysplasia.

20. The method according to claim 18 or claim 19 wherein the non-squamous cellular marker is a marker of cellular proliferation.

21. The method according to claim 18 or claim 19, wherein the non-squamous cellular marker is a marker of columnar cells.

22. The method according to claim 18 or claim 19, wherein the marker is selected from the group consisting of brush border proteins such as villin or moesin, mucin genes, brush border enzymes such as alkaline phosphatase, homeobox genes such as Cdx1 and/or Cdx2, cytokeratins such as CK8/18 for columnar cells, or any marker known to be differentially expressed in Barrett's versus normal oesophageal surface cells.

23. The method according to claim 18 or claim 19, wherein the marker is selected from the group consisting of proliferation markers such as Ki67 and Mcm proteins, proliferation and DNA damage markers such as PCNA, cyclins such as cyclin D and/or cyclin A, abnormal p53, loss of p16, aneuploidy or any marker known to correlate with the degree of dysplasia.

24. The method according to claim 23 wherein the marker is Mcm2 or Cyclin A.

25. The method according to claim 24 wherein Cyclin A is assayed

26. The method according to claim 24 wherein both Mcm2 and Cyclin A are assayed.

27. The method according to claim 18 wherein sampling the cellular surface of the oesophagus comprises the steps of (i) introducing a swallowable device comprising abrasive material capable of collecting cells from the surface of the oesophagus into the subject, (ii) retrieving said device by withdrawal through the oesophagus, and (iii) collecting the cells from the device.

28. The method according to any of claims 18 or 19, further comprising analysing the chromosomal composition of the cells, wherein detection of abnormal karyotype indicates an increased likelihood of dysplasia.

29. The method according to claim 18 or claim 19, further comprising analysing the p53 status of the cells, wherein detection of abnormal p53 status indicates an increased likelihood of dysplasia.

30. The kit according to claim 1 wherein the printed instructions for its use in detection of Barrett's oesophagus or Barrett's associated dysplasia describes a method comprising assaying cells from the surface of a subject's oesophagus for a non-squamous cellular marker, wherein detection of such a marker indicates increased likelihood of the presence of Barrett's or Barrett's associated dysplasia.

31. Use of a capsule sponge in the diagnosis of Barrett's oesophagus or Barrett's associated dysplasia.

32. The method of claim 27, wherein said device comprises a capsule sponge.
